(19) United States
(12) Patent Application Publication
(10) Pub. No.: US 2022/0199248 A1
(43) Pub. Date: Jun. 23, 2022

(54) SYSTEMS AND METHODS FOR NETWORK MONITORING IN REMOTE THERAPY SYSTEMS

(71) Applicant: Advanced Neuromodulation Systems, Inc., Plano, TX (US)

(72) Inventors: Scott DeBates, Frisco, TX (US); Sreetharan Thankathuraipandian, Prosper, TX (US); Doug Lautner, Celina, TX (US); James Nagle, Plano, TX (US)

(21) Appl. No.: 17/132,189

(22) Filed: Dec. 23, 2020

(51) Int. Cl. G16H 40/67; G16H 40/40; G16H 80/00

(52) U.S. Cl. CPC G16H 40/67; G16H 80/00; G16H 40/40

(57) ABSTRACT

The present disclosure provides systems and methods for monitoring network connections in a remote therapy system. A method includes transmitting, at a first time, a polling signal from a first device in the remote therapy system to a second device in the remote therapy system, the polling signal associated with a therapy command, receiving, at a second time, at the first device, a response signal from the second device, the response signal associated with the therapy command, determining a network connectivity status associated with the first device based on a difference between the first time and the second time, and adjusting operation of the remote therapy system based on the determined network connectivity status.

### FIELD OF THE DISCLOSURE

The present disclosure relates generally to remote therapy, and more particularly to monitoring network communications between devices during a remote therapy session.

### BACKGROUND ART

Implantable medical devices have changed how medical care is provided to patients having a variety of chronic illnesses and disorders. For example, implantable cardiac devices improve cardiac function in patients with heart disease by improving quality of life and reducing mortality rates. Further, types of implantable neurostimulators provide a reduction in pain for chronic pain patients and reduce motor difficulties in patients with Parkinson's disease and other movement disorders. In addition, a variety of other medical devices currently exist or are in development to treat other disorders in a wide range of patients.

Many implantable medical devices and other personal medical devices are programmed by a physician or other clinician to optimize the therapy provided by a respective device to an individual patient. The programming may occur using short-range communication links (e.g., inductive wireless telemetry) in an in-person or in-clinic setting.

However, remote patient therapy is a healthcare delivery method that aims to use technology to manage patient health outside of a traditional clinical setting. It is widely expected that remote patient care may increase access to care and decrease healthcare delivery costs.

In at least some known remote therapy systems, a physician is able to program a patient's implantable medical device remotely. Specifically, an audio and/or video conference interface is provided between a patient device and a physician device that allows the physician to remotely assess the patient both before and after adjusting the programming of the patient's implantable medical device.

For example, for a patient with an implanted neuromodulation device, the physician may ask the patient to perform various movement and speech activities as part of the assessment. For a movement assessment, the patient may move away from the patient device (e.g., the patient device may be a mobile computing device that is placed in a desktop cradle during the assessment). This enables the remote physician to view the patient while they perform the requested movement activities.

The implantable medical device may be programmed or otherwise adjusted by the physician by securely communicating with the implantable medical device via the patient device (e.g., over a wireless connection between the patient device and the implantable medical device).

However, at least one challenge with such remote therapy systems is that network reliability has a direct impact on the patient and clinician experience. For example, network issues may prevent a user from successfully initiating a remote therapy session, may cause delayed response times when adjusting remote therapy settings due to network latencies, may result in distorted video or audio due to low network bandwidth, or may result in remote therapy sessions terminating early due to network failure.

Accordingly, it would be desirable to provide a system that monitors and addresses network issues for a remote therapy system.

### BRIEF SUMMARY OF THE DISCLOSURE

In one embodiment, the present disclosure is directed to a method for monitoring network connections in a remote therapy system. The method includes transmitting, at a first time, a polling signal from a first device in the remote therapy system to a second device in the remote therapy system, the polling signal associated with a therapy command, receiving, at a second time, at the first device, a response signal from the second device, the response signal associated with the therapy command, determining a network connectivity status associated with the first device based on a difference between the first time and the second time, and adjusting operation of the remote therapy system based on the determined network connectivity status.

In another embodiment, the present disclosure is directed to a remote therapy system. The remote therapy system includes a first device, and a second device, the first device configured to transmit, at a first time, a polling signal from the first device to the second device, the polling signal associated with a therapy command, receive, at a second time, a response signal from the second device, the response signal associated with the therapy command, determine a network connectivity status associated with the first device based on a difference between the first time and the second time, and adjust operation of the remote therapy system based on the determined network connectivity status.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a diagram of one embodiment of a network environment for implementing remote therapy sessions.

FIG. 2 is one embodiment of a clinician user interface that may be displayed, for example, on the clinician device shown in FIG. 1.

FIG. 3 is one embodiment of a patient user interface that may be displayed, for example, on the patient device shown in FIG. 1.

FIG. 4 is a data flow diagram illustrating one embodiment of network monitoring during a remote therapy session.

FIG. 5 is a data flow diagram illustrating one embodiment of network monitoring during a remote therapy session.

FIG. 6 is a flowchart of one embodiment of a method for performing network monitoring during initialization of a remote therapy session.

FIGS. 7A and 7B are a flowchart of one embodiment of a method for performing network monitoring during a remote therapy session.

FIGS. 8A-8C are a flowchart of one embodiment of a method for performing network monitoring during a remote therapy session.

FIG. 9 is one embodiment of a clinician user interface that may be displayed on the clinician device shown in FIG. 1.

FIG. 10 is a flowchart of one embodiment of a method for updating, at a first device, a network strength level for a second device.

FIG. 11 illustrates one embodiment of a computing device that may be used to implement the systems and methods described herein.

Corresponding reference characters indicate corresponding parts throughout the several views of the drawings.

### DETAILED DESCRIPTION OF THE DISCLOSURE

The present disclosure provides systems and methods for monitoring network connections in a remote therapy system. A method includes transmitting, at a first time, a polling signal from a first device in the remote therapy system to a second device in the remote therapy system, the polling signal associated with a therapy command. The method includes receiving, at a second time, at the first device, a response signal from the second device, the response signal associated with the therapy command. The method further includes determining a network connectivity status associated with the first device based on a difference between the first time and the second time, and adjusting operation of the remote therapy system based on the determined network connectivity status.

As described below, the systems and methods described herein facilitate enhancing remote therapy systems to include network monitoring functionality that notifies users of network issues, dynamically adapts network usage, and reverts to known, safe therapy settings upon a loss of network connectivity.

The network monitoring functionality is initialized, for example, in response to a user initiating a remote therapy session. As part of the network monitoring functionality, the embodiments described herein verify that one or more devices in the system have a working network connection, and confirm that data throughput (e.g., latency) meets a predetermined threshold before the remote therapy session begins. If these criteria are not met, the user is notified, and suggestions on how to address any network issues are provided to the user.

The embodiments described herein also include a heartbeat (or sign of life) functionality. As part of the heartbeat functionality, a heartbeat signal is transmitted between a patient device and a clinician device, as described in more detail below. If the heartbeat signal fails to reach the patient device and/or clinician device repeatedly (e.g., fails to reach the associated device three times in a row), the remote therapy session is terminated and the system reverts to known, safe settings. The patient and clinician are also notified of the network issues and termination of the remote therapy session.

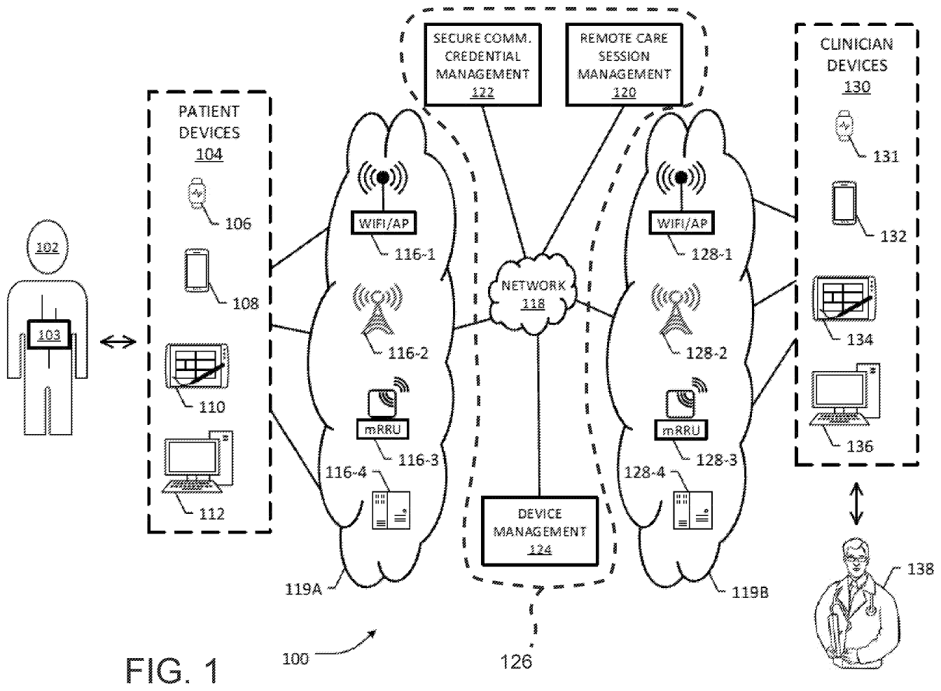

Referring now to the drawings, and in particular to FIG. 1, a network environment is indicated generally at 100. One or more embodiments of a remote care therapy application or service may be implemented in network environment 100, as described herein. In general, “remote care therapy” may involve any care, biomedical monitoring, or therapy that may be provided by a clinician, a medical professional or a healthcare provider, and/or their respective authorized agents (including digital/virtual assistants), with respect to a patient over a communications network while the patient and the clinician/provider are not in close proximity to each other (e.g., not engaged in an in-person office visit or consultation). Accordingly, in some embodiments, a remote care therapy application may form a telemedicine or a telehealth application or service that not only allows healthcare professionals to use electronic communications to evaluate, diagnose and treat patients remotely, thereby facilitating efficiency as well as scalability, but also provides patients with relatively quick and convenient access to diversified medical expertise that may be geographically distributed over large areas or regions, via secure communications channels as described herein.

Network environment 100 may include any combination or sub-combination of a public packet-switched network infrastructure (e.g., the Internet or worldwide web, also sometimes referred to as the “cloud”), private packet-switched network infrastructures such as Intranets and enterprise networks, health service provider network infrastructures, and the like, any of which may span or involve a variety of access networks, backhaul and core networks in an end-to-end network architecture arrangement between one or more patients, e.g., patient(s) 102, and one or more authorized clinicians, healthcare professionals, or agents thereof, e.g., generally represented as caregiver(s) or clinician(s) 138.

Example patient(s) 102, each having a suitable implantable device 103, may be provided with a variety of corresponding external devices for controlling, programming, otherwise (re)configuring the functionality of respective implantable medical device(s) 103, as is known in the art. Such external devices associated with patient(s) 102 are referred to herein as patient devices 104, and may include a variety of user equipment (UE) devices, tethered or untethered, that may be configured to engage in remote care therapy sessions. By way of example, patient devices 104 may include smartphones, tablets or phablets, laptops/desktops, handheld/palmtop computers, wearable devices such as smart glasses and smart watches, personal digital assistant (PDA) devices, smart digital assistant devices, etc., any of which may operate in association with one or more virtual assistants, smart home/office appliances, smart TVs, virtual reality (VR), mixed reality (MR) or augmented reality (AR) devices, and the like, which are generally exemplified by wearable device(s) 106, smartphone(s) 108, tablet(s)/phablet(s) 110 and computer(s) 112. As such, patient devices 104 may include various types of communications circuitry or interfaces to effectuate wired or wireless communications, short-range and long-range radio frequency (RF) communications, magnetic field communications, Bluetooth communications, etc., using any combination of technologies, protocols, and the like, with external networked elements and/or respective implantable medical devices 103 corresponding to patient(s) 102.

With respect to networked communications, patient devices 104 may be configured, independently or in association with one or more digital/virtual assistants, smart home/premises appliances and/or home networks, to effectuate mobile communications using technologies such as Global System for Mobile Communications (GSM) radio access network (GRAN) technology, Enhanced Data Rates for Global System for Mobile Communications (GSM) Evolution (EDGE) network (GERAN) technology, 4G Long Term Evolution (LTE) technology, Fixed Wireless technology, 5th Generation Partnership Project (5GPP or 5G) technology, Integrated Digital Enhanced Network (IDEN) technology, WiMAX technology, various flavors of Code Division Multiple Access (CDMA) technology, heterogeneous access network technology, Universal Mobile Telecommunications System (UMTS) technology, Universal Terrestrial Radio Access Network (UTRAN) technology, All-IP Next Generation Network (NGN) technology, as well as technologies based on various flavors of IEEE 802.11 protocols (e.g., WiFi), and other access point (AP)-based technologies and microcell-based technologies such as femtocells, picocells, etc. Further, some embodiments of patient devices 104 may also include interface circuitry for effectuating network connectivity via satellite communications. Where tethered UE devices are provided as patient devices 104, networked communications may also involve broadband edge network infrastructures based on various flavors of Digital Subscriber Line (DSL) architectures and/or Data Over Cable Service Interface Specification (DOCSIS)-compliant Cable Modem Termination System (CMTS) network architectures (e.g., involving hybrid fiber-coaxial (HFC) physical connectivity). Accordingly, by way of illustration, an edge/access network portion 119A is exemplified with elements such as WiFi/AP node(s) 116-1, macro/microcell node(s) 116-2 and 116-3 (e.g., including micro remote radio units or RRUs, base stations, eNB nodes, etc.) and DSL/CMTS node(s) 116-4.

Similarly, clinicians 138 may be provided with a variety of external devices for controlling, programming, otherwise (re)configuring or providing therapy operations with respect to one or more patients 102 mediated via respective implantable medical device(s) 103, in a local therapy session and/or remote therapy session, depending on implementation and use case scenarios. External devices associated with clinicians 138, referred to herein as clinician devices 130, may include a variety of UE devices, tethered or untethered, similar to patient devices 104, which may be configured to engage in remote care therapy sessions as will be set forth in detail further below. Clinician devices 130 may therefore also include devices (which may operate in association with one or more virtual assistants, smart home/office appliances, VRAR virtual reality (VR) or augmented reality (AR) devices, and the like), generally exemplified by wearable device(s) 131, smartphone(s) 132, tablet(s)/phablet(s) 134 and computer(s) 136. Further, example clinician devices 130 may also include various types of network communications circuitry or interfaces similar to that of patient device 104, which may be configured to operate with a broad range of technologies as set forth above. Accordingly, an edge/access network portion 119B is exemplified as having elements such as WiFi/AP node(s) 128-1, macro/microcell node(s) 128-2 and 128-3 (e.g., including micro remote radio units or RRUs, base stations, eNB nodes, etc.) and DSL/CMTS node(s) 128-4. It should therefore be appreciated that edge/access network portions 119A, 119B may include all or any subset of wireless communication means, technologies and protocols for effectuating data communications with respect to an example embodiment of the systems and methods described herein.

In one arrangement, a plurality of network elements or nodes may be provided for facilitating a remote care therapy service involving one or more clinicians 138 and one or more patients 102, wherein such elements are hosted or otherwise operated by various stakeholders in a service deployment scenario depending on implementation (e.g., including one or more public clouds, private clouds, or any combination thereof). In one embodiment, a remote care session management node 120 is provided, and may be disposed as a cloud-based element coupled to network 118, that is operative in association with a secure communications credentials management node 122 and a device management node 124, to effectuate a trust-based communications overlay/tunneled infrastructure in network environment 100 whereby a clinician may advantageously engage in a remote care therapy session with a patient. Session management node 120, credentials management node 122, and device management node 124 may be implemented, for example, by a backend server 126 (also referred to herein as a remote therapy backend).

In the embodiments described herein, implantable medical device 103 may be any suitable medical device. For example, implantable medical device may be a neurostimulation device that generates electrical pulses and delivers the pulses to nervous tissue of a patient to treat a variety of disorders.

One category of neurostimulation systems is deep brain stimulation (DBS). In DBS, pulses of electrical current are delivered to target regions of a subject's brain, for example, for the treatment of movement and effective disorders such as PD and essential tremor. Another category of neurostimulation systems is spinal cord stimulation (SCS) for the treatment of chronic pain and similar disorders.

Neurostimulation systems generally include a pulse generator and one or more leads. A stimulation lead includes a lead body of insulative material that encloses wire conductors. The distal end of the stimulation lead includes multiple electrodes, or contacts, that intimately impinge upon patient tissue and are electrically coupled to the wire conductors. The proximal end of the lead body includes multiple terminals (also electrically coupled to the wire conductors) that are adapted to receive electrical pulses. In DBS systems, the distal end of the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted in the patient within a subcutaneous pocket created during the implantation procedure.

The pulse generator is typically implemented using a metallic housing (or can) that encloses circuitry for generating the electrical stimulation pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on the proximal end of a stimulation lead.

Although implantable medical device 103 is described in the context of a neurostimulation device herein, those of skill in the art will appreciate that implantable medical device 103 may be any type of implantable medical device.

As discussed above, the systems and methods described herein conduct dynamic network monitoring to notify users of network issues, dynamically adapt network usage, and revert to known, safe settings upon a loss of network connectivity.

To initialize a remote therapy session, in the embodiments described herein, a data throughput is monitored (e.g., by patient device 104, clinician device 130, and/or backend server 126). If the data throughput meets a predetermined threshold, the remote therapy session will begin. Otherwise, if the data throughput does not meet the predetermined threshold, one or more users are notified that there is a network connectivity issue, and suggestions may be provided to the users to address the network connectivity issue.

During operation of an established remote therapy session, the systems and methods described herein also monitor data throughput (e.g., packet latency and bandwidth) of therapy commands transmitted between devices in the remote therapy system to detect network connectivity issues. For example, data throughput of therapy commands may be measured between clinician device 130 and backend server 126, between patient device 104 and backend server 126, and/or between any devices in network environment 100. For example, in some embodiments, data throughput may be alternatively or additionally monitored between patient device 104 and implantable device 103.

Within network environment 100 there are a number of devices in communication with one another, and the round trip time it takes between transmitting a signal from one device to another device (potentially via one or more intermediary devices) and receiving a response signal at the one device from the another device may be monitored to identify communications and/or network issues. Further, the data communications between different pairs of devices in the system must be synchronized with one another. For example, in one embodiment, sensors on implantable device 103 communicate with implantable device 103 based on a first clock, sensors on implantable device 103 communicate with patient device 104 based on a second clock, and sensors on implantable device communicate with backend server 126 based on a third clock. To prevent communications failures, the first, second, and third clocks should all be synchronized with one another.

Based on the detected data throughput, a network strength level, or latency level (e.g., good, okay, poor, no network) is determined and reported to one of more users (e.g., patient 102 and/or clinician 138). Each network strength level is associated with a range of data throughput values. Further, when the network strength level changes (e.g., moving from good to okay), video and/or audio resolutions may be adjusted to improve performance, and one or more users may be notified of the adjustment. In some embodiments, other actions may alternatively or additionally be taken (e.g., routing communications between patient device 104 and clinician device 130 though a different backend server 126).

In some embodiments, at least one device in network environment (e.g., patient device 104, clinician device 130, and/or backend server 126) may prompt a user to approve adjustment of the video and/or audio resolution. For example, if the network strength level changes from okay to poor, clinician device 130 may display a prompt asking clinician 138 to approve reduction of the video and/or audio resolution. Alternatively, adjustments to the video and/or audio resolution may be made automatically.

As noted above, in addition to monitoring therapy commands, transmission and reception of a heartbeat signal may be monitored between devices in network environment 100, as described in more detail below.

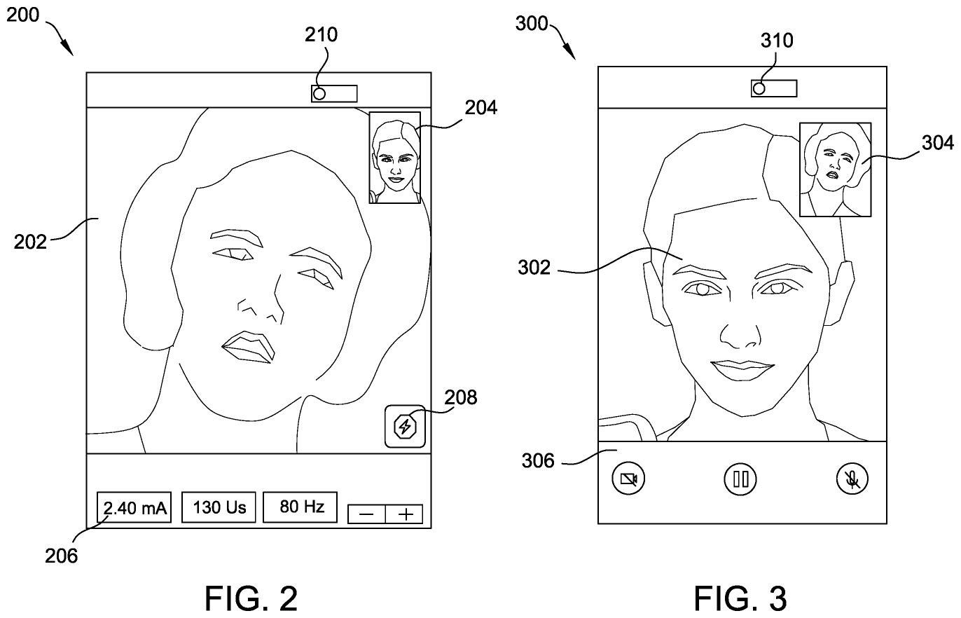

FIG. 2 is one embodiment of a clinician user interface 200 that may be displayed, for example, on clinician device 130 (shown in FIG. 1). Clinician user interface 200 includes a patient video feed 202 (enabling clinician 138 to view patient 102) and a clinician video feed 204 (enabling clinician 138 to view themselves). Further, clinician user interface 200 includes a parameter adjustment panel 206 that enables clinician 138 to remotely adjust parameters for implantable device 103 during the remote therapy session. For example, when implantable device 103 is a neurostimulation device, clinician 138 may adjust an amplitude, a pulse width, and/or a frequency of a stimulation waveform delivered to patient 102 via implantable device 103. Clinician user interface 200 also includes a stop stimulation button 208 that enables clinician 138 to command implantable device 103 to stop applying stimulation to patient.

Further, as shown in FIG. 2, clinician user interface 200 includes a network status indicator 210 that indicates the network strength level (e.g., good, okay, poor, no network). In the embodiment shown, network status indicator 210 includes a circular icon that changes color based on the network strength level (e.g., green for good, yellow for okay, orange for poor, and red for no network). Those of skill in the art will appreciate, however, that network status indicator 210 may include any suitable graphic for conveying the network strength level. For example, in some embodiments, the network strength level may be expressed as a percentage, as a textual message, etc.

FIG. 3 is one embodiment of a patient user interface 300 that may be displayed, for example, on patient device 104 (shown in FIG. 1). Patient user interface 300 includes a clinician video feed 302 (enabling patient 102 to view clinician 138) and a patient video feed 304 (enabling patient 102 to view themselves). Further, patient user interface 300 includes a control panel 306 that enables patient 102 to, for example, end or pause the remote therapy session.

Further, as shown in FIG. 3, patient user interface 300 includes a network status indicator 310 that indicates the network strength level (e.g., good, okay, poor, no network). In the embodiment shown, network status indicator 310 includes a circular icon that changes color based on the network strength level (e.g., green for good, yellow for okay, orange for poor, and red for no network). Those of skill in the art will appreciate, however, that network status indicator 310 may include any suitable graphic for conveying the network strength level. For example, in some embodiments, the network strength level may be expressed as a percentage, as a textual message, etc.

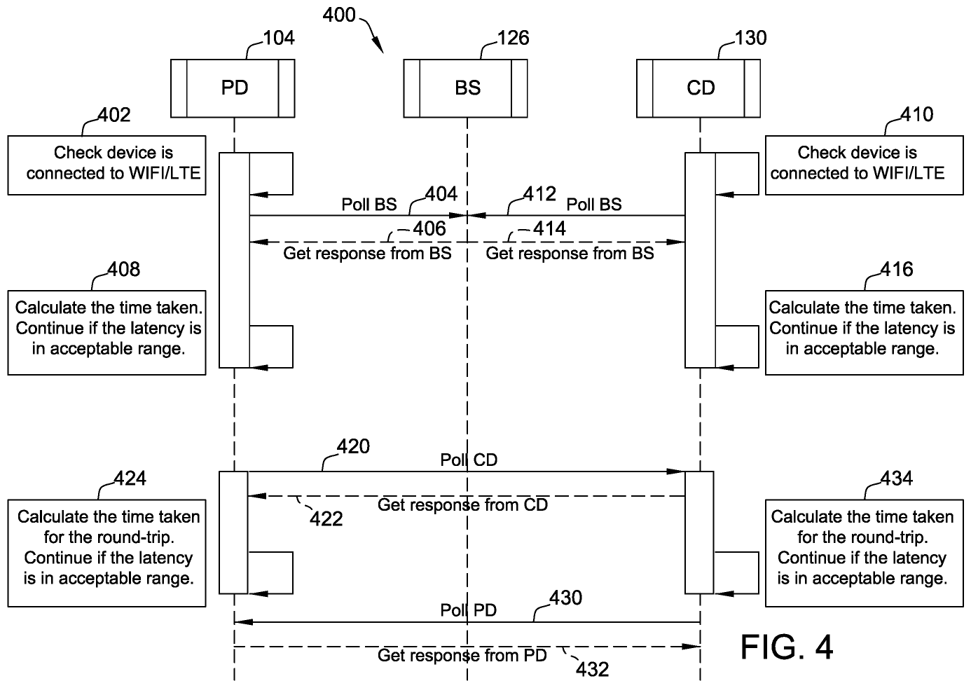

FIG. 4 is a data flow diagram 400 illustrating one embodiment of network monitoring during a remote therapy session. Diagram 400 illustrates data flow between patient device 104, backend server 126, and clinician device 130.

Diagram 400 illustrates several examples of monitoring data throughput between devices in network environment 100. For example, to monitor network issues, in one embodiment, patient device 104 confirms 402 that patient device 104 has a network connection (e.g., a Wifi connection or an LTE connection). One the network connection is confirmed, patient device 104 transmits 404 a polling signal to backend server 126, and receives 406 a response signal from backend server 126. The polling signal may include, for example, data associated with therapy commands and/or any other suitable type of data. Patient device 104 then calculates 408 a latency based on the time taken between transmission 404 and reception 406. If the latency is within an acceptable range (e.g., a predetermined range), the remote therapy session continues. Otherwise, patient device 104 may terminate the remote therapy session.

Similarly, to monitor network issues, clinician device 130 confirms 410 that clinician device 130 has a network connection (e.g., a Wifi connection or an LTE connection). One the network connection is confirmed, clinician device 130 transmits 412 a polling signal to backend server 126, and receives 414 a response signal from backend server 126. The polling signal may include, for example, data associated with therapy commands and/or any other suitable type of data. Clinician device 130 then calculates 416 a latency based on the time taken between transmission 412 and reception 414. If the latency is within an acceptable range (e.g., a predetermined range), the remote therapy session continues. Otherwise, clinician device 130 may terminate the remote therapy session.

Further, as shown in FIG. 4, to monitor network issues, patient device 104 transmits 420 a polling signal to clinician device 130, and receives 422 a response signal from clinician device 130. The polling signal may include, for example, data associated with therapy commands and/or any other suitable type of data. Patient device 104 then calculates 424 a latency based on the time taken between transmission 420 and reception 422. If the latency is within an acceptable range (e.g., a predetermined range), the remote therapy session continues. Otherwise, patient device 104 may terminate the remote therapy session.

Similarly, to monitor network issues, in the embodiment shown, clinician device 130 transmits 430 a polling signal to patient device 104, and receives 432 a response signal from patient device 104. The polling signal may include, for example, data associated with therapy commands and/or any other suitable type of data. Clinician device 130 then calculates 434 a latency based on the time taken between transmission 430 and reception 432. If the latency is within an acceptable range (e.g., a predetermined range), the remote therapy session continues. Otherwise, clinician device 130 may terminate the remote therapy session.

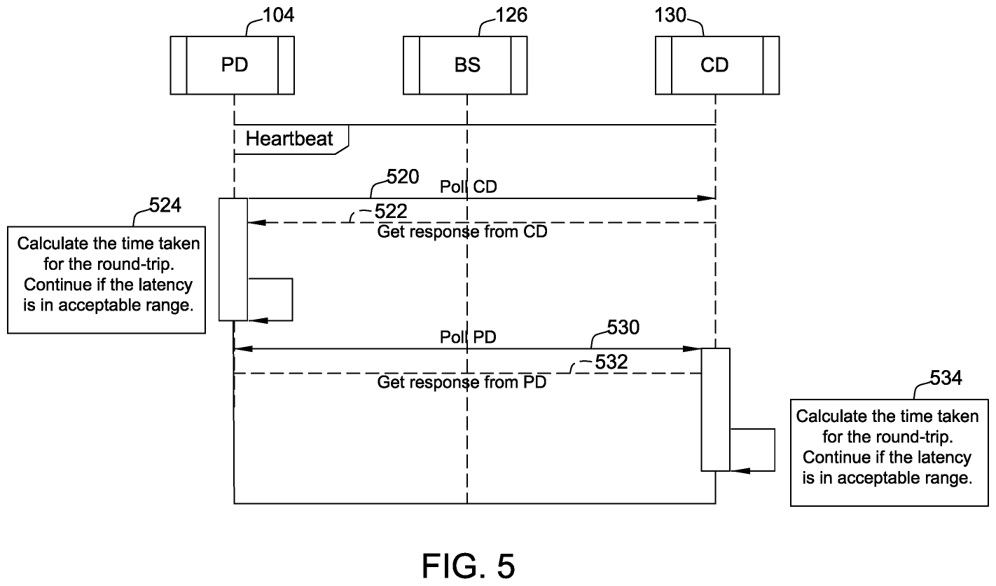

FIG. 5 is a data flow diagram 500 illustrating one embodiment of network monitoring during a remote therapy session. Diagram 500 illustrates data flow for a heartbeat functionality between patient device 104 and clinician device 130. Those of skill in the art will appreciate that similar a heartbeat functionality may be utilized between different pairs of devices in network environment 100.

As shown in FIG. 5, to monitor network issues, patient device 104 transmits 520 a heartbeat polling signal to clinician device 130, and receives 522 a heartbeat response signal from clinician device 130. The heartbeat polling signal may be transmitted 520, for example, periodically (e.g., every 10 seconds), to repeatedly confirm throughout the remote therapy session that patient device 104 is communicatively coupled to clinician device 130. That is, if patient device 104 repeatedly transmits 520 the heartbeat polling signal but does not receive a heartbeat response signal, patient device 104 may determine that the network connection between patient device 104 and clinician device 130 has failed.

The heartbeat polling signal may include, for example, data associated with therapy commands and/or any other suitable type of data. Patient device 104 then calculates 524 a latency based on the time taken between transmission 520 and reception 522. If the latency is within an acceptable range (e.g., a predetermined range), the remote therapy session continues. Otherwise, patient device 104 may terminate the remote therapy session.

Similarly, to monitor network issues, in the embodiment shown, clinician device 130 transmits 530 a heartbeat polling signal to patient device 104, and receives 532 a heartbeat response signal from patient device 104. The heartbeat polling signal may be transmitted 520, for example, periodically, to periodically confirm that clinician device 130 is communicatively coupled to patient device 104. That is, if clinician device 130 repeatedly transmits 530 the heartbeat polling signal but does not receive a heartbeat response signal, clinician device 130 may determine that the network connection between patient device 104 and clinician device 130 has failed.

The polling signal may include, for example, data associated with therapy commands and/or any other suitable type of data. Clinician device 130 then calculates 534 a latency based on the time taken between transmission 530 and reception 532. If the latency is within an acceptable range (e.g., a predetermined range), the remote therapy session continues. Otherwise, clinician device 130 may terminate the remote therapy session.

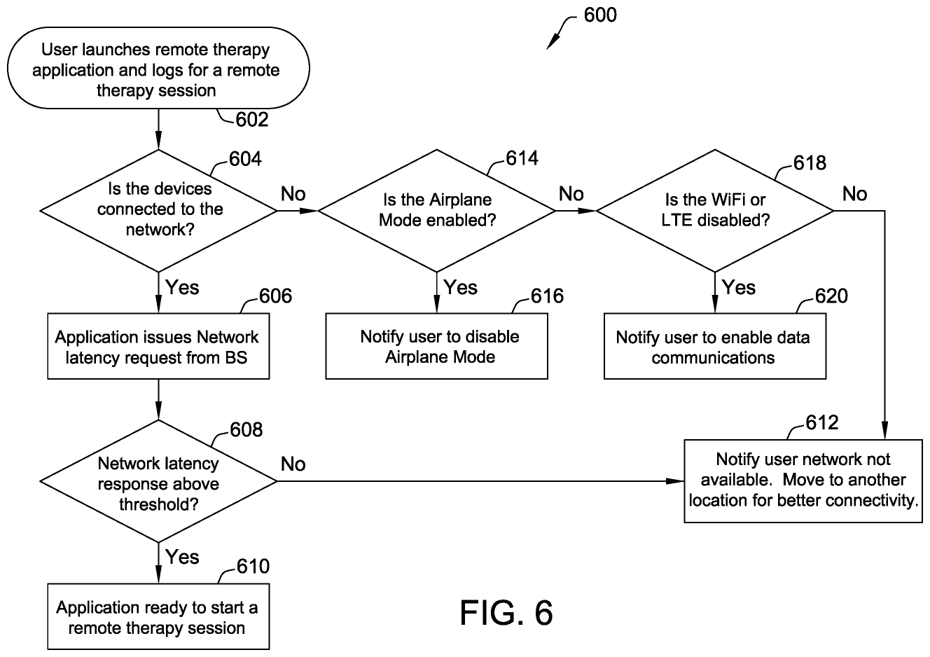

FIG. 6 is a flowchart of one embodiment of a method 600 for performing network monitoring during initialization of a remote therapy session. Method 600 may be performed, for example, by patient device 103 or clinician device 130. FIG. 6 begins at block 602, when a user launches a remote therapy application (e.g., patient 102 launches a remote therapy session on patient device 104 or clinician 130 launches a remote therapy session on clinician device 130) and logs in to the remote therapy session.

At block 604, it is determined whether the device is connected to the network. If so, flow proceeds to block 606, and the device issues a network latency request from backend server 126 and determines a network latency response (e.g., by transmitting a polling signal to backend server 126 and receiving a response signal from backend server 126). Flow continues to block 608, and it is determined whether the network latency response is above a predetermined threshold. If the network latency response is above the predetermined threshold, flow proceeds to block 610, and the remote therapy application indicates to the user that the remote therapy session is ready to be initiated. If the network latency response is not above the predetermined threshold, flow proceeds to block 612, and the user is notified that the network is not available and prompted to move to a different location to attempt to improve connectivity.

Returning to block 604, if it is determined that the device is not connected to the network, flows proceeds to block 614, and it is determined whether an airplane mode (e.g., in which device is prevented from establishing a network connection) is currently enabled on the device. If airplane mode is activated, flow proceeds to block 616, and the device notifies the user to disable airplane mode. Otherwise, flow proceeds to block 618.

At block 618, it is determined whether network connectivity (e.g., WiFi or LTE connectivity) is currently disabled. If so, flow proceeds to block 620, and the device notifies the user to enable network connectivity. Otherwise, flow proceeds to block 612, and the user is notified that the network is not available and prompted to move to a different location to attempt to improve connectivity.

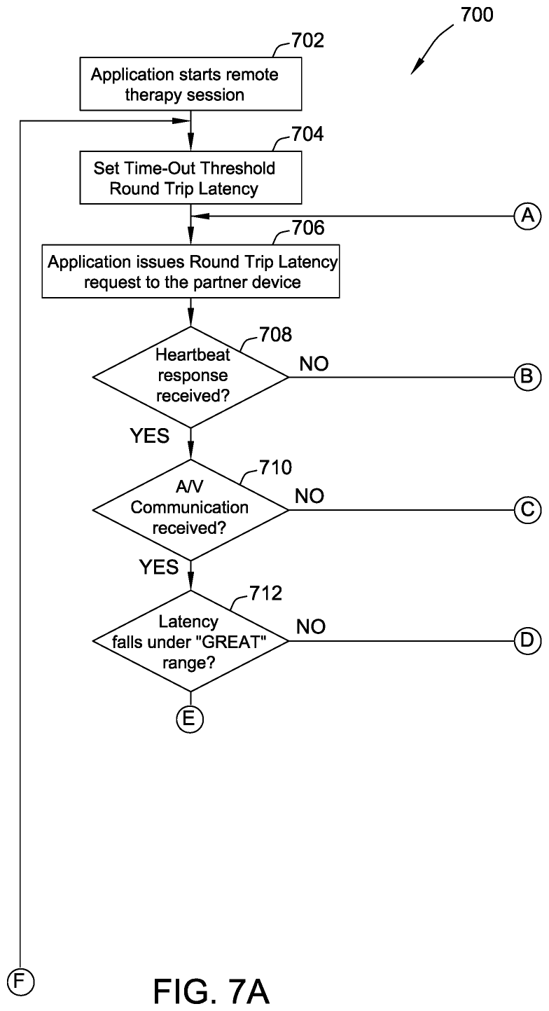

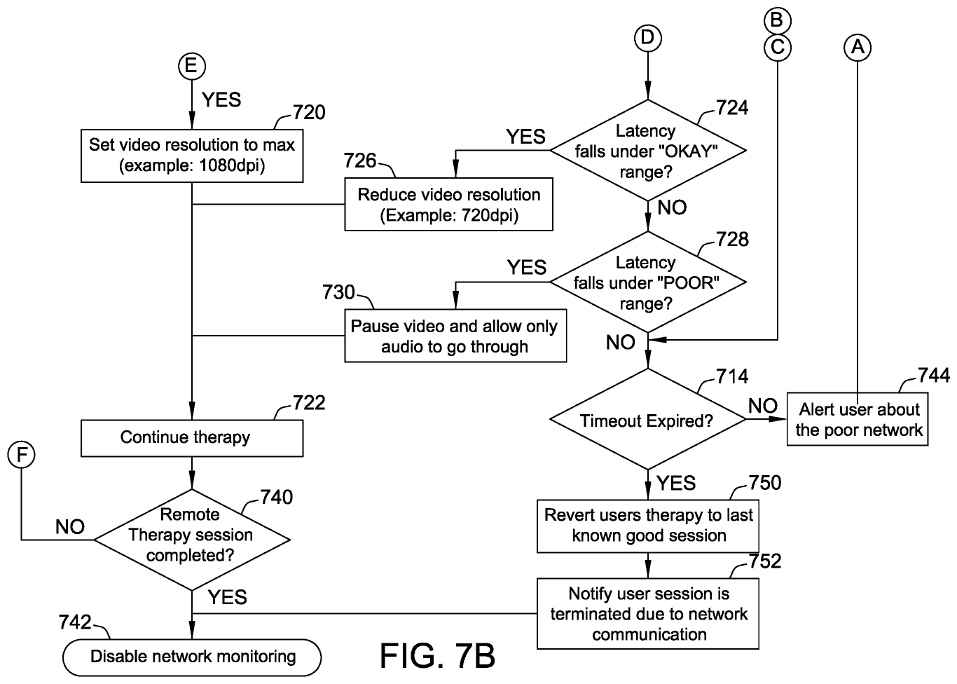

FIGS. 7A and 7B are a flowchart of one embodiment of a method 700 for performing network monitoring during a remote therapy session. Method 700 may be performed, for example, by patient device 103. At block 702, the device (via the remote therapy application) initiates the remote therapy session (e.g., after completion of method 600 (shown in FIG. 6)).

From block 702, flow proceeds to block 704, where a time out threshold for round trip latency monitoring is set. For example, the device may terminate the remote therapy session if the device sends a number of heartbeat polling signals exceeding the time out threshold without receiving corresponding heartbeat return signals. Flow then proceeds to block 706.

At block 706, a round trip latency request is initiated by the device (e.g., by sending a polling signal to another device over the network). For example, patient device 104 may send a polling signal to clinician device 130. As part of the round trip latency request, the device may receive a response signal and calculate a latency level based on the time difference between the transmission of the polling signal and the receipt of the response signal.

In this embodiment, flow proceeds to block 708, where the devices determines whether a heartbeat response signal has been received (i.e., in response to the most recently transmitted heartbeat polling signal). If the heartbeat response signal is received, flow proceeds to block 710, and the device determines whether an audio/video communication has been received. If the audio/video communication has been received, flow proceeds to block 712. If, at block 710, no audio/video communication has been received, or if, at block 708 no heartbeat response signal has been received, flow proceeds to block 714 (described below).

In relation to the audio/video communication, the audio and video functionality of the remote therapy session may use compression/synchronization algorithms such as H264, or AAC to reduce latencies. In one embodiment, video data for clinician 130 and patient 102 is encoded into packets (e.g., H264) that are encrypted (e.g., using transport layer security) and transmitted to a backend system hosting the remote therapy session (e.g., backend server 126). The remote therapy session validates that data received is from a valid user (e.g., when the remote therapy session is initiated, a token is provided to the applications operating on patient device 104 and clinician device 130 that enables access to the remote therapy session for sharing and receiving encoded packages). Upon the remote therapy session validating that a packet is from a valid user, the packet is routed to all parties connected to the session. The applications operating on patient device 104 and clinician device 130 will obtain the packets, decode the packets, and display corresponding image on the associated device.

For example, video and audio of clinician 138 is captured using clinician device 130 (e.g., using a microphone and camera), and send as encoded packets with a token to backend server 126. Backend server 126 authenticates the token, and routes the encoded packets to patient device 104. The encoded packets are decoded at patient device 104, with audio data from the packets routed to speakers on patient device 104, and video data from the packets routed to a display buffer on patient device 104. A similar process occurs to transmits audio and video data from patient device 104 to clinician device 130.

During transmission and receipt of such packets, packet loss, packet error, and/or packet retransmission may occur. By monitoring for such events (e.g., packet loss, packet, error, packet retransmission), it may be determined whether the network bandwidth is acceptable for the current video/audio resolution. Setting thresholds for the number of events (e.g., packet loss, packet, error, packet retransmission), the resolution can be lowered when the thresholds are reached, reducing such errors.

Returning back to FIG. 7, at block 712, the device determines whether the calculated latency falls within a “GREAT” range. Although latency ranges of “GREAT”, “OKAY”, and “POOR” are described in connection with method 700, those of skill in the art will appreciate that any suitable latency categorizations may be used. If the calculated latency falls within the “GREAT” range, flow proceeds to block 720, and the video resolution for the therapy session is set to a maximum possible value (e.g., 1080p). Flow then proceeds to block 722, and the remote therapy session continues.

If the calculated latency does not fall within the “GREAT” range, flow proceeds to block 724, and the device determines whether the calculated latency falls within the “OKAY” range. If so, flow proceeds to block 726, where the video resolution is reduced (e.g., to 720p), before flow proceeds to block 722.

If the calculated latency does not fall within the “OK” range, flow proceeds to block 728, and the device determines whether the calculated latency falls within the “POOR” range. If so, flow proceeds to block 730, where video functionality is inhibited (allowing patient 102 and clinician 138 to still communicate via audio), before flow proceeds to block 722. If the calculated latency does not fall within the “OK” range, flow proceeds to block 714.

Returning back to block 722, flow proceeds to block 740, and the device determines whether the remote therapy session is complete. If the session is not complete, flow returns to block 704. If the session is complete, flow proceeds to block 742 and network monitoring for the remote therapy session is terminated.

Returning to block 714, the device determines whether the time out threshold set at block 704 has been exceeded. If not, the user is alerted that the network connection is poor at block 744, and flow returns to block 706. If the time out threshold has been exceeded, flow proceeds to block 750.

At block 750, in advance of terminating the remote therapy session, the therapy settings for the implantable device 103 are set to predetermined values known to be safe to patient 102. The “safe” settings may be therapy settings previously used by patient 102 (e.g., the therapy settings used immediately prior to establishment of the remote therapy session), or may be therapy settings selected by clinician 138 (e.g., using clinician device 130). Alternatively, the “safe” settings may be any other suitable set of therapy settings that will not negatively impact the patient.

From block 750, flow proceeds to block 752, at which the remote therapy session is terminated and the user is notified that the remote therapy session is being terminated due to network connectivity failures. Flow then proceeds to block 742 and network monitoring for the remote therapy session is terminated.

Those of skill in the art will appreciate that method 700 is only one example of monitoring network functionality during a remote therapy session, and that various modifications may be made to method 700 within the spirit and scope of the present disclosure.

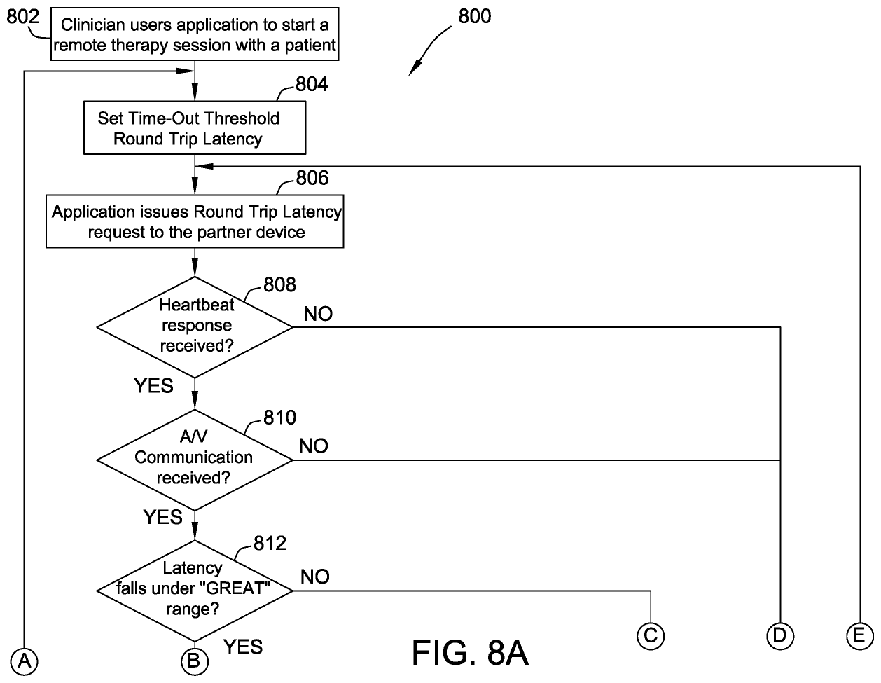

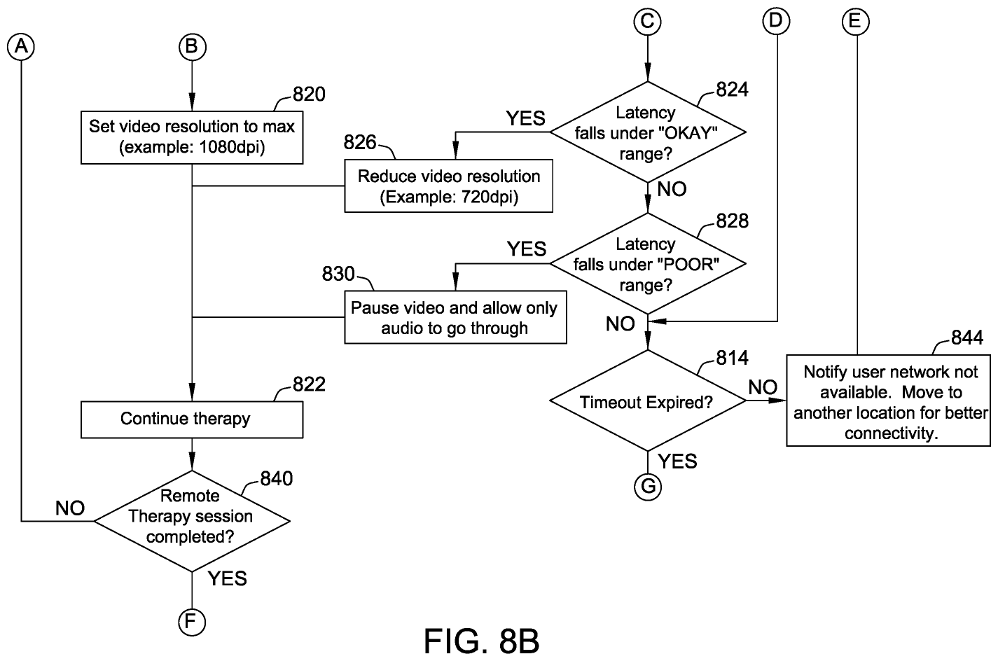

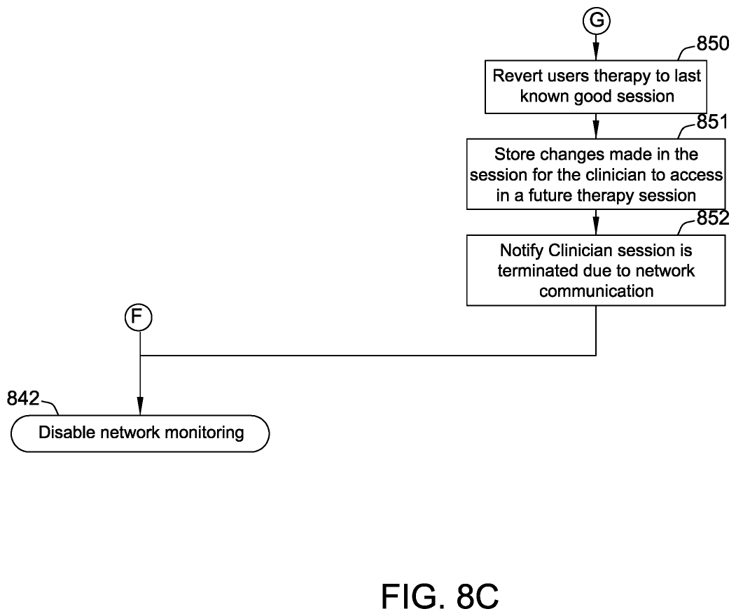

FIGS. 8A-8C are a flowchart of one embodiment of a method 800 for performing network monitoring during a remote therapy session. Method 800 may be performed, for example, by clinician device 130. At block 802, the device (via the remote therapy application) initiates the remote therapy session (e.g., after completion of method 600 (shown in FIG. 6)).

From block 802, flow proceeds to block 804, where a time out threshold for round trip latency monitoring is set. For example, the device may terminate the remote therapy session if the device sends a number of heartbeat polling signals exceeding the time out threshold without receiving corresponding heartbeat return signals. Flow then proceeds to block 806.

At block 806, a round trip latency request is initiated by the device (e.g., by sending a polling signal to another device over the network). For example, clinician device 130 may send a polling signal to patient device 104. As part of the round trip latency request, the device may receive a response signal and calculate a latency level based on the time difference between the transmission of the polling signal and the receipt of the response signal.

In this embodiment, flow proceeds to block 808, where the devices determines whether a heartbeat response signal has been received (i.e., in response to the most recently transmitted heartbeat polling signal). If the heartbeat response signal is received, flow proceeds to block 810, and the device determines whether an audio/video communication has been received, as discussed above. If the audio/video communication has been received, flow proceeds to block 812. If, at block 810, no audio/video communication has been received, or if, at block 808 no heartbeat response signal has been received, flow proceeds to block 814 (described below).

At block 812, the device determines whether the calculated latency falls within a “GREAT” range. Although latency ranges of “GREAT”, “OKAY”, and “POOR” are described in connection with method 800, those of skill in the art will appreciate that any suitable latency categorizations may be used. If the calculated latency falls within the “GREAT” range, flow proceeds to block 820, and the video resolution for the therapy session is set to a maximum possible value (e.g., 1080p). Flow then proceeds to block 822, and the remote therapy session continues.

If the calculated latency does not fall within the “GREAT” range, flow proceeds to block 824, and the device determines whether the calculated latency falls within the “OKAY” range. If so, flow proceeds to block 826, where the video resolution is reduced (e.g., to 720p), before flow proceeds to block 822.

If the calculated latency does not fall within the “OK” range, flow proceeds to block 828, and the device determines whether the calculated latency falls within the “POOR” range. If so, flow proceeds to block 830, where video functionality is inhibited (allowing patient 102 and clinician 138 to still communicate via audio), before flow proceeds to block 822. If the calculated latency does not fall within the “OK” range, flow proceeds to block 814.

Returning back to block 822, flow proceeds to block 840, and the device determines whether the remote therapy session is complete. If the session is not complete, flow returns to block 804. If the session is complete, flow proceeds to block 842 and network monitoring for the remote therapy session is terminated.

Returning to block 814, the device determines whether the time out threshold set at block 804 has been exceeded. If not, the user is alerted that the network connection is poor at block 844, and flow returns to block 806. If the time out threshold has been exceeded, flow proceeds to block 850.

At block 850, in advance of terminating the remote therapy session, the therapy settings for the implantable device 103 are set to predetermined values known to be safe to patient 102. The “safe” settings may be therapy settings previously used by patient 102 (e.g., the therapy settings used immediately prior to establishment of the remote therapy session), or may be therapy settings selected by clinician 138 (e.g., using clinician device 130). Alternatively, the “safe” settings may be any other suitable set of therapy settings that will not negatively impact the patient.

From block 850, flow proceeds to block 851, and any therapy changes made during the remote therapy are stored for potential future use by clinician 138. Flow then proceeds to block 852, at which the remote therapy session is terminated and clinician 138 is notified that the remote therapy session is being terminated due to network connectivity failures. Flow then proceeds to block 842 and network monitoring for the remote therapy session is terminated.

Those of skill in the art will appreciate that method 800 is only one example of monitoring network functionality during a remote therapy session, and that various modifications may be made to method 800 within the spirit and scope of the present disclosure.

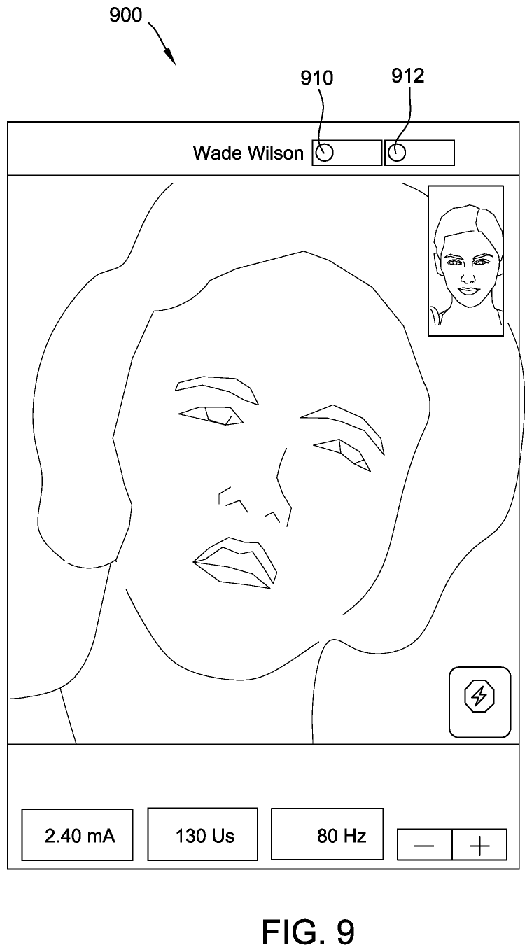

In one embodiment, network status indicators for multiple devices may be displayed on a single device (e.g., patient device 104 and/or clinician device 130). For example, FIG. 9 is one embodiment of a clinician user interface 900 that may be displayed, for example, on clinician device 130 (shown in FIG. 1). Clinician user interface 900 is substantially similar to clinician user interface 200 (shown in FIG. 2), except that clinician user interface 900 includes a first network status indicator 910 that indicates the network strength level (e.g., good, okay, poor, no network) for clinician device 130, as well as a second network status indicator 912 that indicates the network strength level (e.g., good, okay, poor, no network) for clinician device patient device 104. In the embodiment shown, network status indicators 910 and 912 each include a circular icon that changes color based on the network strength level (e.g., green for good, yellow for okay, orange for poor, and red for no network). Those of skill in the art will appreciate, however, that network status indicators 910 and 912 may each include any suitable graphic for conveying the network strength level. For example, in some embodiments, the network strength level may be expressed as a percentage, as a textual message, etc. Patient device 104 may similarly include two different network status indicators.

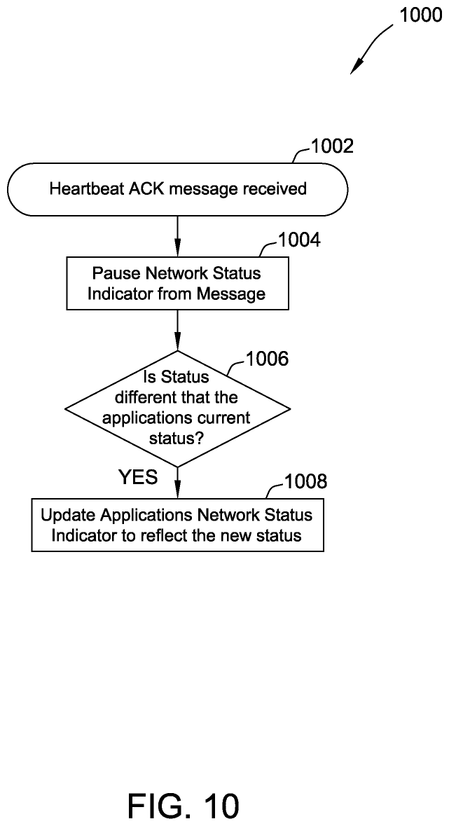

The network strength level for one device (e.g., patient device 104) may be communicated to another device (e.g., clinician device 130) via a heartbeat response signal (described above). For example, FIG. 10 is a flowchart of one embodiment of a method 1000 for updating, at a first device, a network strength level for a second device.

Specifically, at block 1002, the first device receives a heartbeat response message from the second device. Flow proceeds to block 1004, and the network strength level for the second device is parsed from the heartbeat response message. That is, the network strength level for the second device is included in the heartbeat response message.

Flow then proceeds to block 1006, and the first device determines whether the parsed network strength level is different that the network strength level for the second device currently stored on the first device (e.g., as part of the remote therapy application). If so, flow proceeds to block 1008, and the network strength level for the second device stored on the first device is updated to the parsed network strength level. Otherwise, no change is made to the stored network strength level.

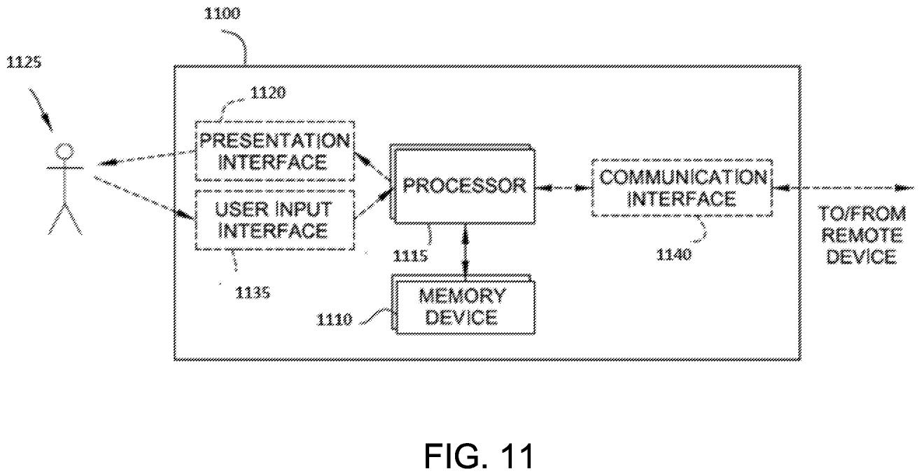

FIG. 11 illustrates one embodiment of a computing device 1100 that may be used to implement the systems and methods described herein. For example, computing device 1100 may be used to implement patient device 104, backend server 126, and/or clinician device 130 (both shown in FIG. 1).

Computing device 1100 includes at least one memory device 1110 and a processor 1115 that is coupled to memory device 1110 for executing instructions. In some embodiments, executable instructions are stored in memory device 1110. In this embodiment, computing device 1100 performs one or more operations described herein by programming processor 1115. For example, processor 1115 may be programmed by encoding an operation as one or more executable instructions and by providing the executable instructions in memory device 1110.

Processor 1115 may include one or more processing units (e.g., in a multi-core configuration). Further, processor 1115 may be implemented using one or more heterogeneous processor systems in which a main processor is present with secondary processors on a single chip. In another illustrative example, processor 1115 may be a symmetric multi-processor system containing multiple processors of the same type. Further, processor 1115 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein. In one embodiment, processor 1115 is a GPU (as opposed to a central processing unit (CPU)). Alternatively, processor 1115 may be any processing device capable of implementing the systems and methods described herein.

In this embodiment, memory device 1110 is one or more devices that enable information such as executable instructions and/or other data to be stored and retrieved. Memory device 1110 may include one or more computer readable media, such as, without limitation, dynamic random access memory (DRAM), static random access memory (SRAM), a solid state disk, and/or a hard disk. Memory device 1110 may be configured to store, without limitation, application source code, application object code, source code portions of interest, object code portions of interest, configuration data, execution events and/or any other type of data. In one embodiment, memory device 1110 is a GPU memory unit. Alternatively, memory device 1110 may be any storage device capable of implementing the systems and methods described herein.

In this embodiment, computing device 1100 includes a presentation interface 1120 that is coupled to processor 1115. Presentation interface 1120 presents information to a user 1125 (e.g., patient 102 or physician 138). For example, presentation interface 1120 may include a display adapter (not shown) that may be coupled to a display device, such as a cathode ray tube (CRT), a liquid crystal display (LCD), an organic LED (OLED) display, and/or an “electronic ink” display. In some embodiments, presentation interface 1120 includes one or more display devices.

In this embodiment, computing device 1100 includes a user input interface 1135. User input interface 1135 is coupled to processor 1115 and receives input from user 1125. User input interface 1135 may include, for example, a keyboard, a pointing device, a mouse, a stylus, a touch sensitive panel (e.g., a touch pad or a touch screen), a gyroscope, an accelerometer, a position detector, and/or an audio user input interface. A single component, such as a touch screen, may function as both a display device of presentation interface 1120 and user input interface 1135.

Computing device 1100, in this embodiment, includes a communication interface 1140 coupled to processor 1115. Communication interface 1140 communicates with one or more remote devices. To communicate with remote devices, communication interface 440 may include, for example, a wired network adapter, a wireless network adapter, and/or a mobile telecommunications adapter.

The embodiments described herein provide systems and methods for monitoring network connections in a remote therapy system. A method includes transmitting, at a first time, a polling signal from a first device in the remote therapy system to a second device in the remote therapy system, the polling signal associated with a therapy command. The method includes receiving, at a second time, at the first device, a response signal from the second device, the response signal associated with the therapy command. The method further includes determining a network connectivity status associated with the first device based on a difference between the first time and the second time, and adjusting operation of the remote therapy system based on the determined network connectivity status.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A method for monitoring network connections in a remote therapy system, the method comprising:
transmitting, at a first time, a polling signal from a first device in the remote therapy system to a second device in the remote therapy system, the polling signal associated with a therapy command;
receiving, at a second time, at the first device, a response signal from the second device, the response signal associated with the therapy command;
determining a network connectivity status associated with the first device based on a difference between the first time and the second time; and
adjusting operation of the remote therapy system based on the determined network connectivity status.

2. The method of claim 1, wherein adjusting operation of the remote therapy system comprises adjusting at least one of an audio quality and a video quality of a remote therapy session established between a patient device and a clinician device.

3. The method of claim 1, wherein adjusting operation of the remote therapy system comprises causing an indication of the determined network connectivity status to be displayed on the first device.

4. The method of claim 3, further comprising causing an additional network connectivity status associated with the second device to be displayed on the first device.

5. The method of claim 1, wherein adjusting operation of the remote therapy system comprises terminating a previously established remote therapy session.

6. The method of claim 1, further comprising:
periodically transmitting heartbeat signals from the first device; and
assessing network connectivity of the first device based on whether or not the first device receives heartbeat response signals in response to the periodically transmitted heartbeat signals.

7. The method of claim 1, wherein the first device is a patient device, a clinician device, or a backend server communicatively coupled between the patient device and the clinician device.

8. The method of claim 1, wherein the second device is a patient device, a clinician device, or a backend server communicatively coupled between the patient device and the clinician device.

9. The method of claim 1, wherein adjusting operation of the remote therapy system comprises prompting a user to take one or more actions to improve network connectivity.

10. The method of claim 1, wherein adjusting operation of the remote therapy system comprises controlling an implantable device to implement known, safe therapy settings.

11. A remote therapy system comprising:
a first device; and
a second device, the first device configured to:
transmit, at a first time, a polling signal from the first device to the second device, the polling signal associated with a therapy command;
receive, at a second time, a response signal from the second device, the response signal associated with the therapy command;
determine a network connectivity status associated with the first device based on a difference between the first time and the second time; and
adjust operation of the remote therapy system based on the determined network connectivity status.

12. The system of claim 11, wherein to adjust operation of the remote therapy system, the first device is configured to adjust at least one of an audio quality and a video quality of a remote therapy session established between a patient device and a clinician device.

13. The system of claim 11, wherein to adjust operation of the remote therapy system, the first device is configured to display an indication of the determined network connectivity status on the first device.

14. The system of claim 13, wherein the first device is further configured to display an additional network connectivity status associated with the second device on the first device.

15. The system of claim 11, wherein to adjust operation of the remote therapy system, the first device is configured to terminate a previously established remote therapy session.

16. The system of claim 11, wherein the first device is further configured to:
periodically transmit heartbeat signals from the first device; and
assess network connectivity of the first device based on whether or not the first device receives heartbeat response signals in response to the periodically transmitted heartbeat signals.

17. The system of claim 11, wherein the first device comprises a patient device, a clinician device, or a backend server communicatively coupled between the patient device and the clinician device.

18. The system of claim 11, wherein the second device comprises a patient device, a clinician device, or a backend server communicatively coupled between the patient device and the clinician device.

19. The system of claim 11, wherein to adjust operation of the remote therapy system, the first device is configured to prompt a user to take one or more actions to improve network connectivity.

20. The system of claim 11, wherein to adjust operation of the remote therapy system, the first device is configured to control an implantable device to implement known, safe therapy settings.
